(19) United States
(12) Patent Application Publication
(10) Pub. No.: US 2010/0099116 A1
(43) Pub. Date: Apr. 22, 2010

(54) Methods for Analysis of Hedgehog Pathway Inhibitors

(75) Inventors: Kerrie L. Faia, Topsfield, MA (US); Everton Mandley, Dorchester, MA (US); Wei Niu, Wayland, MA (US); Christine Pien, Topsfield, MA (US)

Correspondence Address: Foley Hoag, LLP (w/IPX), 155 Seaport Blvd., Boston, MA, 02210, US

(73) Assignee: Infinity Discovery, Inc., Cambridge, MA (US)

(21) Appl. No.: 12/521,079

(22) PCT Filed: Jan. 14, 2008
(86) PCT No.: PCT/US08/50970

§ 371 Date: Oct. 14, 2009

Related U.S. Application Data
(60) Provisional application No. 60/880,211, filed on Jan. 12, 2007.

(51) Int. Cl. G01N 33/53; G01N 33/68; C12Q 1/48

(52) U.S. Cl. 435/7.1; 436/86; 435/15

(57) ABSTRACT

One aspect of the present invention relates to a method of ascertaining the inhibitory activity in a mammal of a candidate inhibitor of the hedgehog pathway. In certain embodiments, the candidate inhibitor is administered systemically. In certain embodiments, the mammal is a rodent or primate. In certain embodiments, the mammal is a mouse. In certain embodiments, the candidate inhibitor is a small molecule or natural product.

### RELATED APPLICATIONS

This application claims the benefit of priority to U.S. Provisional Patent Application Ser. No. 60/880,211, filed Jan. 12, 2007; the entirety of which is hereby incorporated by reference.

### BACKGROUND OF THE INVENTION

Members of the Hedgehog family of signaling molecules mediate many important short- and long-range patterning processes during invertebrate and vertebrate development. Exemplary hedgehog genes and proteins are described in PCT publications WO 95/18856 and WO 96/17924 (both of which are hereby incorporated by reference). The vertebrate family of hedgehog genes includes at least four members, three of which, herein referred to as Desert hedgehog (Dhh), Sonic hedgehog (Shh) and Indian hedgehog (Ihh), apparently exist in all vertebrates, including fish, birds, and mammals. A fourth member, herein referred to as tiggie-winkle hedgehog (Thh), appears to be specific to fish. Desert hedgehog (Dhh) is expressed principally in the testes, both in mouse embryonic development and in the adult rodent and human; Indian hedgehog (Ihh) is involved in bone development during embryogenesis and in bone formation in the adult; and Shh is primarily involved in morphogenic and neuroinductive activities.

Hedgehog (Hh) has been identified as a tumor growth signal in a diversity of human cancers (Berman, et al., Nature, 425:846-851, 2003; Karhadkar, et al., Nature, 431:707-712, 2004). Tumors originating in the esophagus, stomach, biliary tract, pancreas and prostate express high levels of Shh and Ihh, which stimulate the growth of tumor cells. Functional neutralization antibodies against Shh and Ihh have been shown to block the growth of these tumors in vitro and in xenografts, establishing that these tumors are dependent on Hh ligand for their growth. The ability to modulate one or more genes that are part of the hedgehog signaling cascade thus represents a possible therapeutic approach to several clinically significant cancers.

A need therefore exists for methods and compounds that inhibit signal transduction activity by modulating activation of a hedgehog, patched, or smoothened-mediated signal transduction pathway, such as the Hedgehog signaling pathway, to reverse or control aberrant growth. In addition, although methods for screening and determining the activity of hedgehog pathway inhibitors are known (for example, U.S. Patent Application No. 2007/0212712, which is hereby incorporated by reference), the need exists to identify additional efficient and accurate in vivo assays for hedgehog pathway inhibitors.

### SUMMARY OF THE INVENTION

Certain aspects of the present invention relate to methods of identifying and determining the inhibitory activity of a candidate inhibitor in a mammal.

One aspect of the invention relates to a method of ascertaining the inhibitory activity of a candidate inhibitor of the hedgehog pathway, comprising the following steps:

inducing an anagen phase in one or more hair follicles in a first mammal and a second mammal;

administering to said first mammal said candidate inhibitor;

measuring a detectable marker of hedgehog pathway activity in said first mammal, thereby obtaining a first detectable marker activity;

measuring said detectable marker of hedgehog pathway activity in said second mammal, thereby obtaining a second detectable marker activity; and

comparing said first detectable marker activity and said second detectable marker activity. A finding of decreased detectable marker activity in said first mammal as compared to said second mammal indicates the inhibitory activity of said candidate inhibitor.

In certain embodiments, the invention relates to the aforementioned method, wherein said candidate inhibitor is administered systemically.

In certain embodiments, the invention relates to the aforementioned method, wherein the anagen phase is induced by chemical or physical depilation.

In certain embodiments, the invention relates to the aforementioned method, wherein the anagen phase is induced by chemical depilation.

In certain embodiments, the invention relates to the aforementioned method, wherein the detectable marker is HhIP, Gli1, Gli2, Gli3, Ptc1, Ptc2, sonic hedgehog, indian hedgehog, or desert hedgehog.

In certain embodiments, the invention relates to the aforementioned method, wherein the detectable marker is measured by RT-PCR, in situ hybridization or immunohistochemistry.

In certain embodiments, the invention relates to the aforementioned method, wherein the mammal is a rodent or primate.

In certain embodiments, the invention relates to the aforementioned method, wherein the mammal is a mouse.

In certain embodiments, the invention relates to the aforementioned method, wherein the mammal is a C57BL/6 or C3HMCA mouse.

In certain embodiments, the invention relates to the aforementioned method, wherein the mouse is a C57BL/6 mouse.

In certain embodiments, the invention relates to the aforementioned method, wherein the anagen phase is induced from about 7 weeks to about 12 weeks after said mammal is born.

One aspect of the invention relates to a method of ascertaining the inhibitory activity of a candidate inhibitor of the hedgehog pathway, comprising the following steps:

artificially inducing an anagen phase in substantially all hair follicles in a section of skin of a first mouse and a second mouse;

administering to said first mouse said candidate inhibitor;

measuring a detectable marker of hedgehog pathway activity in said first mouse, thereby obtaining a first detectable marker activity;

measuring said detectable marker of hedgehog pathway activity in said second mouse, thereby obtaining a second detectable marker activity; and

comparing said first detectable marker activity and said second detectable marker activity. A finding of decreased detectable marker activity in said first mouse as compared to said second mouse indicates the inhibitory activity of said candidate inhibitor.

In certain embodiments, the invention relates to the aforementioned method, wherein said candidate inhibitor is administered systemically.

In certain embodiments, the invention relates to the aforementioned method, wherein the anagen phase is artificially induced by chemical or physical depilation.

In certain embodiments, the invention relates to the aforementioned method, wherein the anagen phase is artificially induced by chemical depilation.

In certain embodiments, the invention relates to the aforementioned method, wherein the detectable marker is HhIP, Gli1, Gli2, Gli3, Ptc1, Ptc2, sonic hedgehog, indian hedgehog, or desert hedgehog.

In certain embodiments, the invention relates to the aforementioned method, wherein the detectable marker is measured by RT-PCR, in situ hybridization or immunohistochemistry.

In certain embodiments, the invention relates to the aforementioned method, wherein the mouse is a C57BL/6 or C3HMCA mouse.

In certain embodiments, the invention relates to the aforementioned method, wherein the mouse is a C57BL/6 mouse.

In certain embodiments, the invention relates to the aforementioned method, wherein the anagen phase is artificially induced from about 7 weeks to about 12 weeks after said mouse is born.

One aspect of the invention relates to a method of ascertaining the inhibitory activity of a candidate inhibitor of the hedgehog pathway, comprising the following steps:

providing a first non-human mammal and a second non-human mammal about a few days before the start of a naturally occurring anagen phase in one or more hair follicles or during a naturally occurring anagen phase of one or more hair follicles;

administering to said first non-human mammal said candidate inhibitor;

measuring a detectable marker of hedgehog pathway activity in said first non-human mammal, thereby obtaining a first detectable marker activity;

measuring said detectable marker of hedgehog pathway activity in said second non-human mammal, thereby obtaining a second detectable marker activity; and

comparing said first detectable marker activity and said second detectable marker activity. A finding of decreased detectable marker activity in said first non-human mammal as compared to said second non-human mammal indicates the inhibitory activity of said candidate inhibitor.

In certain embodiments, the invention relates to the aforementioned method, wherein said candidate inhibitor is administered systemically.

In certain embodiments, the invention relates to the aforementioned method, wherein the detectable marker is HhIP, Gli1, Gli2, Gli3, Ptc1, Ptc2, sonic hedgehog, indian hedgehog, or desert hedgehog.

In certain embodiments, the invention relates to the aforementioned method, wherein the detectable marker is measured by RT-PCR, in situ hybridization or immunohistochemistry.

In certain embodiments, the invention relates to the aforementioned method, wherein said first non-human mammal is a mouse; and said second non-human mammal is a mouse.

In certain embodiments, the invention relates to the aforementioned method, wherein the non-human mammal is a C57BL/6 or C3HMCA mouse.

In certain embodiments, the invention relates to the aforementioned method, wherein the non-human mammal is a C57BL/6 mouse.

In certain embodiments, the invention relates to any one of the aforementioned methods, wherein the candidate inhibitor is administered by inhalation, orally, intravenously, sublingually, ocularly, transdermally, topically, rectally, vaginally, intramuscularly, intra-arterially, intrathecally, subcutaneously, buccally, or nasally.

In certain embodiments, the invention relates to any one of the aforementioned methods, wherein the candidate inhibitor is a small molecule or natural product.

In certain embodiments, the invention relates to any one of the aforementioned methods, wherein the candidate inhibitor is a small molecule with a molecular weight of less than or equal to about 500 amu.

In certain embodiments, the invention relates to any one of the aforementioned methods, wherein the candidate inhibitor is a small molecule with a molecular weight of less than or equal to about 350 amu.

### BRIEF DESCRIPTION OF THE FIGURES

FIG. 1 depicts photographs demonstrating the use of depilation-induced hair growth as a model to study the Hedgehog pathway.

FIG. 2 depicts a graph showing that Gli-1 expression in the skin is upregulated post depilation, as opposed to post shaving.

FIG. 3 depicts graphs showing that [a] Compound 1, [b] Compound 2 and [c] Compound 3 can maintain pathway inhibition out to 48 hours post dose.

FIG. 4 depicts graphs showing that inhibition of Gli-1 by [a] Compound 1, [b] Compound 2 and [c] Compound 3 is dose-responsive.

FIG. 5 depicts a graph showing that Gli-1 levels during natural anagen are also inhibited by Compound 1.

FIG. 6 depicts photographs showing that melanogenesis and hair re-growth post-depilation are prevented by daily administration of 40 mg/kg Compound 1.

FIG. 7 depicts photographs showing that melanogenesis and hair re-growth post-depilation are prevented by BID administration of 6 mg/kg of the SHH blocking antibody 5E1.

FIG. 8 depicts a graph showing that both Compound 1 and the SHH blocking antibody 5E1 inhibit Gli-1 induction on day 16 post depilation.

### DETAILED DESCRIPTION OF THE INVENTION

The Hedgehog cell signaling pathway is normally active during embryonic development and plays a critical role in controlling the growth and differentiation of pluripotent progenitor cells in many tissues, including the skin. In some mammals, such as mice, hair growth cycles are more or less synchronized and each follicle goes through three distinct growth phases: growing (anagen), transitional (catagen), and resting phase (telogen).

The hedgehog pathway is active during the anagen stage of hair follicle development. The inhibitory effect of a candidate inhibitor may be determined by exposure of skin containing one or more hair follicles in the anagen phase with a candidate inhibitor, measuring a detectable marker of hedgehog pathway activity in said skin containing one or more treated hair follicles, and comparing said measured activity of said skin containing one or more treated hair follicles with the measured activity of a detectable marker of hedgehog activity in one or more untreated skin containing hair follicles.

The transition of hair follicles from the telogen phase to the anagen phase can occur as the result of natural hair follicle cycling or can be induced artificially, for example by depilation or treatment with a hedgehog pathway agonist.

The growth cycle of hair follicles of many species of mammals have been studied and can be mapped out. For example, it has been found that the Sonic Hedgehog ligand (SHH) is a key regulator of hair follicle growth and cycling and serves as a switch between the resting (telogen) and the growth (anagen) stage of the hair cycle. The hair cycle in the C57BL/6 mouse has been extensively characterized and post depilation provides a highly standardized model in which to explore Hedgehog pathway biology. Specifically, the hair follicles of C57BL/6 mice at about 2 weeks after gestation enter a catagen phase, at about week 3 enter a telogen phase, at about week 4 enter an anagen phase, at about week 6 enter a catagen phase, and at about week 7 enter a telogen phase. The week 7 telogen phase lasts until about week 12 after gestation.

Transition of the hair follicle into the anagen phase from the telogen phase of follicle growth may also be induced artificially by treatment of the hair follicle with a hedgehog pathway agonist. Likewise, depilation of hair follicles in the telogen phase also induces the hair follicles to enter the anagen stage of the hair follicle growth cycle. Depilation may be physical or chemical. Depilation is meant to include any method of hair removal that induces a hair follicle to cycle into the anagen phase from the telogen phase. Depilation includes but is not limited to treatment with chemical depilatory agents (i.e., mercaptan salts, thioglycolate salts, and hydroxide salts), plucking, waxing, sugaring, pulsed light, or electrograhy. See, for example, U.S. Patent Application 2006/0034952, which is hereby incorporated by reference.

In some instances, the anagen phase is induced by depilation at about weeks 7, at about 8 weeks, at about 9 weeks, at about 10 weeks, at about 11 weeks, at about 12 weeks, at about 13 weeks, or at about 14 weeks after the birth of the mammal.

At around the time of the onset of a natural anagen phase (about around 12 weeks after gestation) or anytime during the anagen phase of a C57BL/6 mouse, two mice (or two groups of mice) are selected and a candidate inhibitor of the hedgehog pathway is administered to one mouse. At some time, post administration of the candidate inhibitor, the activity of the hedgehog pathway in a sample of skin containing one or more hair follicles of each mouse is measured and the activity of the hedgehog pathway in the mouse treated with the candidate inhibitor is compared to the untreated mouse. A decrease in activity of the hedgehog pathway in the treated mouse as compared to the untreated mouse indicates that the candidate inhibitor had an inhibitory affect on the activity of hedgehog pathway in the treated mouse.

In some instances, a candidate inhibitor of the hedgehog pathway is administered to a mammal about 10 days, about 8 days, about 6 days, about 2 days, about 1 day before natural cycling into the anagen phase or about, about 1 day, about 3 days, about 5 days, or about 7 days after the beginning of a naturally occurring anagen phase.

During the telogen phase (about around 7 weeks after gestation) of a C57BL/6 mouse, two mice (or two groups of mice) are selected and a section of skin is depilated on each mouse. A candidate inhibitor of the hedgehog pathway is administered to one mouse. At some time, post administration of the candidate inhibitor, the activity of the hedgehog pathway in a sample of skin containing the hair follicles from the depilated area of each mouse is measured and the activity of the hedgehog pathway in the mouse treated with the candidate inhibitor is compared to the untreated mouse. A decrease in activity of the hedgehog pathway in the treated mouse as compared to the untreated mouse indicates that the candidate inhibitor had an inhibitory affect on the activity of hedgehog pathway in the treated mouse.

In some instances, a candidate inhibitor of the hedgehog pathway is administered to a mammal about 10 days, about 8 days, about 6 days, about 2 days, about 1 day before artificial induction of anagen phase or about, about 1 day, about 3 days, about 5 days, or about 7 days after artificial induction of the anagen phase.

Hedgehog pathway activity may be measured after a single dose of the candidate inhibitor or multiple doses.

Candidate inhibitors may be administered to the mammal by inhalation, topically, orally, intravenously, sublingually, ocularly, transdermally, topically, rectally, vaginally, intramuscularly, intra-arterially, intrathecally, subcutaneously, buccally, or nasally.

Hedgehog pathway activity may be measured in any number of ways known to those of ordinary skill in the art. One method for determining hedgehog pathway activity is by measuring the relative induction of any hedgehog pathway transcription target gene. Transcription target genes include, but are not limited to hedgehog interacting protein (Hhip), Gli1, Gli2, Gli3, patch 1 (Ptc1), and patch 2 (Ptc2). Gene transcription can be measured using real time polymerase chain reaction (RT-PCR) or in situ hybridization. Gene transcription in skin containing one or more hair follicles from treated and untreated mammal groups is compared and a decrease in the gene transcription of hedgehog pathway target genes in the treated group as compared to the untreated group indicates that the hedgehog pathway in the treated group has been inhibited in the skin containing the one or more hair follicles. Likewise, the protein levels of proteins synthesized as a result of the activation of hedgehog pathway target genes may be measured using immunohistochemistry or western blotting.

The candidate inhibitors of the present invention may be selected from small molecule libraries and other libraries including combinatorial chemical libraries. Such libraries are known in the art and are available commercially. Additionally, proprietary libraries are also available for use from collaborators and others. Additionally, the synthesis and screening of small molecule libraries (e.g., combinatorial chemical libraries) are well known in the art (See, for example, U.S. Pat. No. 6,060,596 to Lerner; U.S. Pat. No. 6,185,506 to Cramer, et al.; U.S. Pat. No. 6,377,895 to Horlbeck; U.S. Pat. No. 6,936,477 to Still, et al.; Shipps, et al., Proc. Natl. Acad. Sci. USA, 94:11833-11838, 1997; Stockwell, et al., Chemistry & Biology, 6:71-83, 1999, all of which are incorporated herein by reference; see, also, for example, www.combichem.net; www.combichemistry.com; www.combinatorial.com and pubs.acs.org/journals/jcchff/).

Candidate inhibitor encompass numerous chemical classes, though typically they are organic molecules, preferably small organic compounds having a molecular weight of more than 50 and less than about 3,500 daltons. Candidate reagents comprise functional groups necessary for structural interaction with proteins, particularly hydrogen bonding and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In certain embodiments, the candidate inhibitor may be a small molecule, natural product, antibody, or RNAi.

Candidate inhibitors are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

A candidate inhibitor of the hedgehog pathway can target any pathway member, which leads to a decrease in activity of the pathway. Examples of pathway targets include, but are not limited to, smoothened, hedgehog, patched, Gli-1, and suppressor of fused.

The mammal may be a primate, rodent, canine, feline, ovine, bovine, or ferret. The mammal may be a human.

While several embodiments of the present invention are described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

### EXEMPLIFICATION

The invention now being generally described, it will be more readily understood by reference to the following examples, which are included merely for purposes of illustration of certain aspects and embodiments of the present invention, and are not intended to limit the invention.

As described below, one approach to determine if up-regulation of Hedgehog target genes occurred during depilation-induced anagen utilized 7 week old C57BL/6 mice which were in the telogen phase of the hair cycle, wherein anagen and subsequently hair re-growth were initiated via chemical depilation with Nair®. As a result of anagen initiation, the hair follicle cycles were synchronized allowing for reproducible measurement of Hedgehog target gene expression over time in the skin. Shaved skin, containing hair follicles which remain in telogen, served as a control. From days 6 through 14 post depilation, corresponding to mid- to late-anagen, Hedgehog target gene expression was measured by RT-PCR. It was shown that the Hedgehog signaling pathway is active during anagen as SHH, Gli-1, Gli-2 and PTCH-1 were all up-regulated in the depilated but not the shaved-skin samples. The highest level of Hedgehog target gene expression was noted on day 10 post depilation. Smoothened (SMO) levels remained constant throughout the study and did not differ between telogen and anagen.

Having established the up-regulation of Hedgehog target genes during depilation-induced anagen, novel SMO antagonists, such as Compound 1, were evaluated in this model. Compound 1 is an orally bioavailable cyclopamine derivative with favorable PK properties and is a potent inhibitor of the Hedgehog pathway. To test compound activity, a single oral dose of either vehicle or Compound 1 was administered on day 10 post depilation. At various time points post dose, both shaved and depilated skin samples were collected to evaluate gene expression. In a dose-proportional manner, Compound 1 completely inhibited GLI-1 up-regulation in the depilated skin as early as 8 hours post dose and maintained complete inhibition out to 48 hours. Compound 1 also inhibited GLI-1 expression induced as a result of natural anagen, which occurs at approximately 12 weeks of age. Of note, after hair follicle synchronization, onset of melanogenesis occurred on day 9 post depilation and hair re-growth by day 14. Daily administration of Compound 1 or BID administration of the SHH blocking antibody 5E1 inhibited hair re-growth post depilation. Collectively, this data suggests that Hedgehog target gene expression and regulation in the hair follicle offers an attractive biomarker for Hedgehog pathway antagonists under evaluation in the clinic as anti-cancer agents.

### Example 1

### Use of Depilation to Control the Hair Cycle

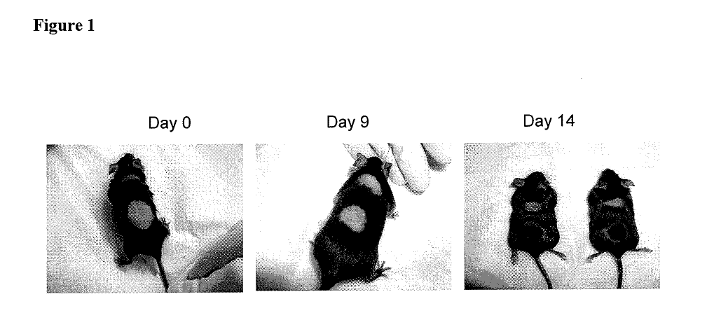

The use of depilation induced hair growth as a model to study the Hedgehog pathway is shown in FIG. 1. The anagen phase of fur growth was induced in 7 week old, male C57/BL6 mice (Taconic, Germantown, N.Y.) by depilation with Nair® (Church and Dwight Co., Inc. Princeton, N.J. 08543). Prior to depilation, mice were temporarily anesthetized via an intraperitoneal (i.p.) injection of a ketamine/zylazine cocktail. Mice were then shaved on their dorsal side, Nair® was applied topically with a plastic spatula and allowed to remain on the mouse skin for 2 minutes. The Nair® and fur were gently removed by washing the depilation area with water. An area of skin from each mouse was also shaved to provide a non-depilation control. By day 9 post depilation, the skin was pigmented indicating the onset of follicular melanogenesis. Fur had completely grown back by day 14 on the area of Nair® treated skin, while the shaved skin, still in telogen, remains fur free.

### Example 2

### Measuring Gli-1 Expression

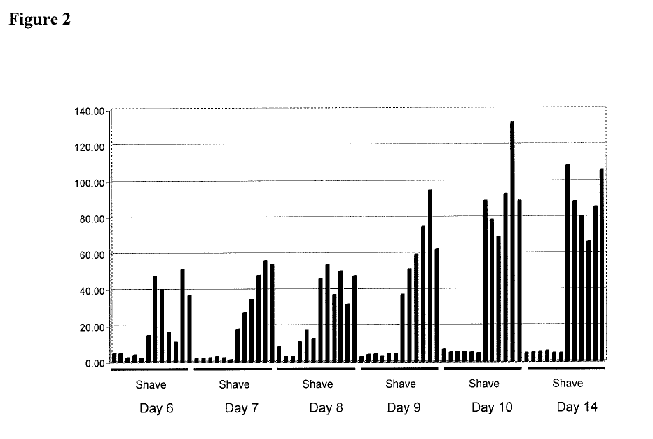

Gli-1 expression in the skin is upregulated post depilation as shown in FIG. 2. Mice were shaved and treated with Nair®; on days 6, 7, 8, 9, 10 and 14 post shaving/depilation, the mice were sacrificed and their skin collected for RT-PCR analysis. RNA was isolated from the mouse skin using Trizol Reagent (Invitrogen). Total RNA was DNAse treated using the RNeasy kit (Qiagen) and gene expression analysis for various Hedgehog family members performed by single step quantitative RT-PCR using the Applied Biosystems 7300 real time PCR machine, One-Step Master Mix and Taqman gene expression assays. Relative gene quantification was determined by following the delta CT method described by Applied Biosystems; GAPDH was used as the internal control.

FIG. 2 shows the expression level of GLI-1 in the shaved and depilated skin samples throughout the study. Gli-1, Gli-2, and SHH were all upregulated in the depilated, but not in the shaved skin samples, indicating that the Hedgehog signaling pathway is active during anagen but not telogen. The highest level of Gli-1 expression was achieved on day 10 post depilation. SMO expression was also measured and remained constant throughout the study.

### Example 3

### Effect of Compound 1, Compound 2 and Compound 3 on Gli-1 Expression

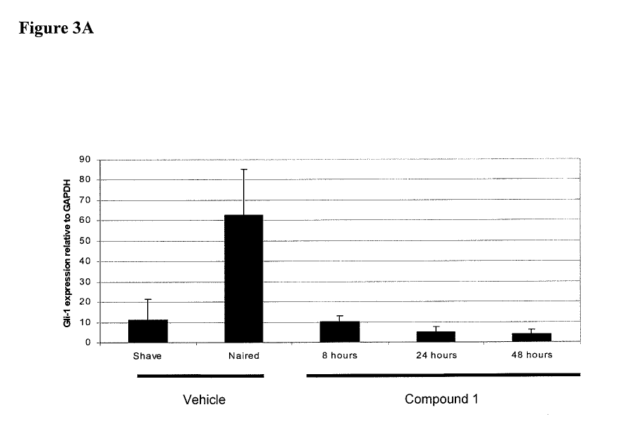

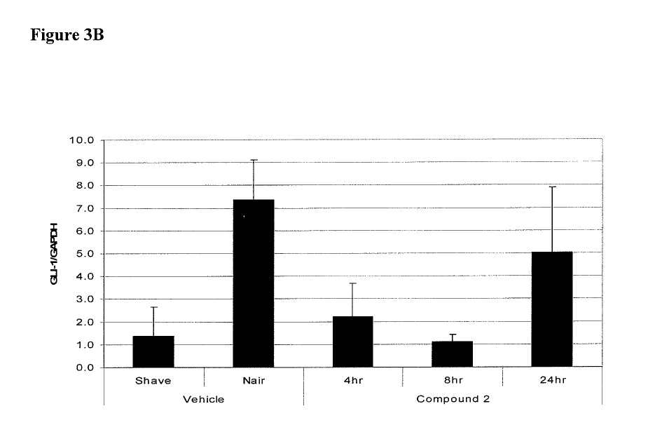

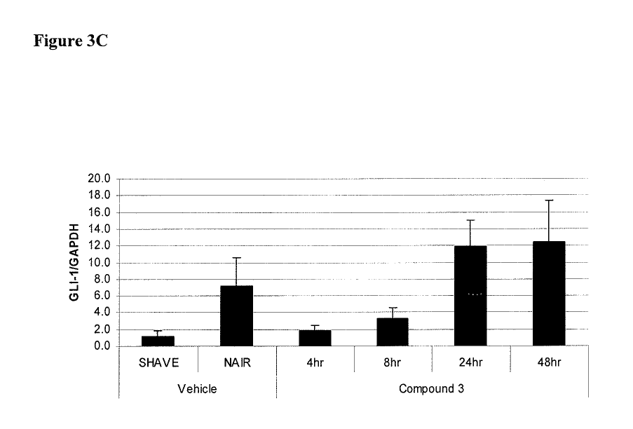

It was then shown that Compound 1, Compound 2 and Compound 3 can maintain pathway inhibition out to 48 hours post dose (see FIG. 3). Mice were given a single oral dose of either 100 mg/kg Compound 1, 40 mg/kg Compound 2, or 10 mg/kg Compound 3, all diluted in a vehicle of 30% HBPCD, or vehicle alone on day 10 post depilation. At various time points post dose (4, 8, 24 or 48 hours post dose), the mice were sacrificed and the skin collected for RT-PCR analysis. Both shaved and depilated skin was collected from the vehicle treated animals as controls. As a result of depilation, Gli-1 expression in the skin was elevated approximately 6 fold compared to levels obtained in the shaved skin. Complete Gli-1 inhibition was achieved by 8 hours post Compound 1 administration and no recovery was seen out to 48 hours. Therefore, a single oral dose of Hedgehog pathway antagonist, Compound 1, was able to completely inhibit maximally active Hedgehog pathway as a result of anagen synchronization caused by depilation. With Compound 2 dosed at 40 mg/kg and Compound 3 dosed at 10 mg/kg, Gli-1 expression was maximally reduced by 4-8 hours and fully recovered by 24 hours post dose.

### Example 4

### Dose Responsiveness of the Effect of Compound 1, Compound 2 and Compound 3 on Gli-1 Inhibition

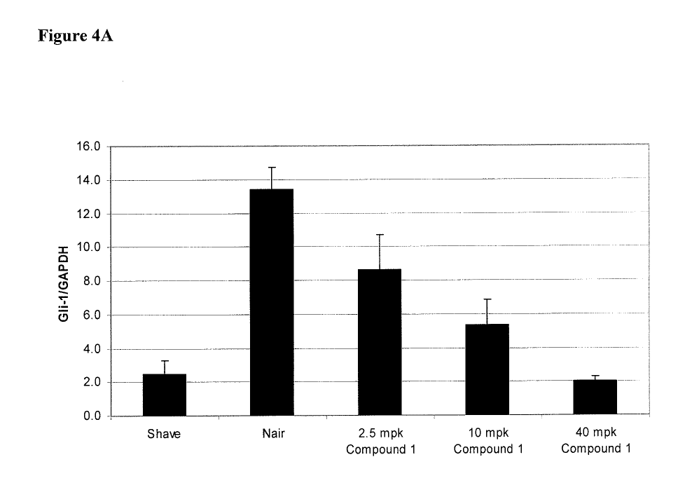

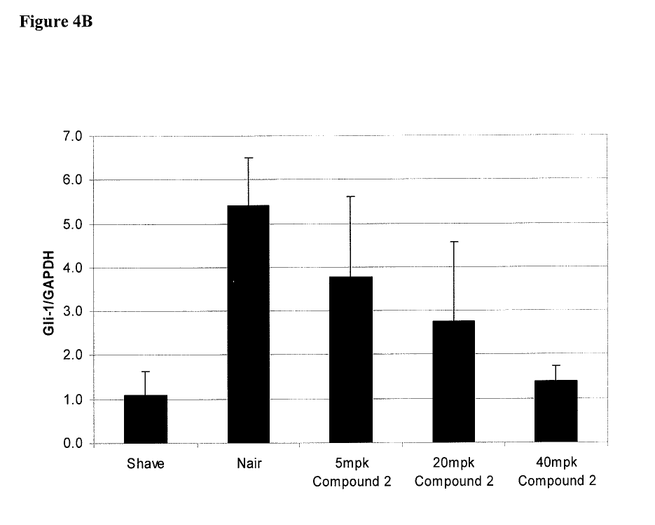

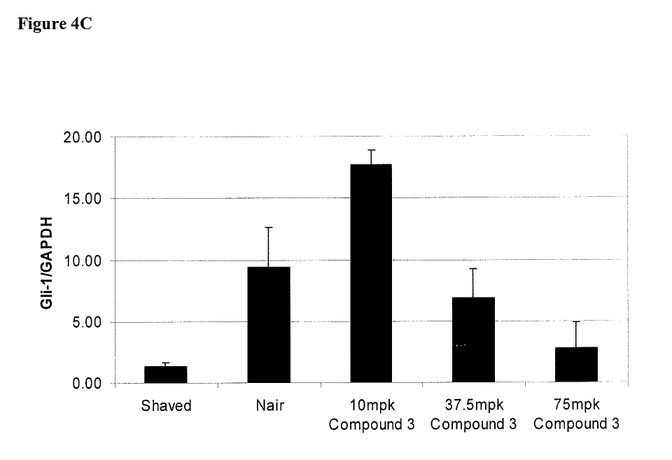

Mice were given a single oral dose of Vehicle (5% HPBCD) or Compound 1 at either 2.5, 10 or 40 mg/kg on day 10 post depilation. All skin samples were harvested at 8 hours post dose. RT-PCR on the shaved and Nair®-treated skin samples from the vehicle-treated mice showed an approximate 6 fold upregulation of Gli-1 expression as a result of depilation. This upregulation of Gli-1 was inhibited in a dose responsive manner by Compound 1, as shown in FIG. 4a.

Mice were given a single oral dose of Vehicle (5% HPBCD) or Compound 3 at either 10, 37.5 or 75 mg/kg on day 10 post depilation. All skin samples were harvested at 24 hours post dose. RT-PCR on the shaved and Nair®-treated skin samples from the vehicle-treated mice showed an approximate 6 fold upregulation of Gli-1 expression as a result of depilation. This upregulation of Gli-1 was inhibited in a dose responsive manner by Compound 3, as shown in FIG. 4b.

Mice were given a single oral dose of Vehicle (5% HPBCD) or Compound 2 at either 5, 20 or 40 mg/kg on day 10 post depilation. All skin samples were harvested at 8 hours post dose. RT-PCR on the shaved and Nair®-treated skin samples from the vehicle-treated mice showed an approximate 6 fold upregulation of Gli-1 expression as a result of depilation. This upregulation of Gli-1 was inhibited in a dose responsive manner by Compound 2, as shown in FIG. 4c.

### Example 5

### Effect of Compounds on the Inhibition of Gli-1 Levels During Natural Anagen

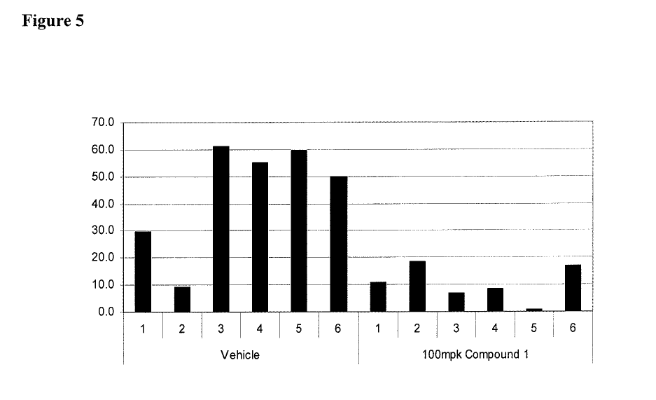

It was further shown that Gli-1 levels during natural anagen were also inhibited by Compound 1 (see FIG. 5). As mentioned above, the hair cycle in the C57BL/6 mouse has been extensively characterized and it has been published that between 7-12 weeks of age the hair follicle is in its resting stage, known as telogen. Therefore, to determine if Compound 1 could inhibit Gli-1 expression caused by the onset of naturally occurring anagen, mice were shaved during telogen and observed daily for the first signs of melanogeneis, which occurred anywhere from 12-14 weeks of age with the first signs of hair re-growth post shaving at 13-15 weeks of age. At the first signs of melanogeneis the mice were randomized into 2 groups and were dosed with either vehicle or 100 mg/kg Compound 1. Skin was collected 8 hours post dose. As shown in FIG. 5, Gli-1 levels in the skin were lower in the mice treated with Compound 1 compared to vehicle, indicating that Compound 1 inhibited hedgehog pathway activity during naturally occurring anagen.

### Example 6

### Effect of Blocking Hedgehog Activity on Hair Re-Growth Post Depilation During Anagen

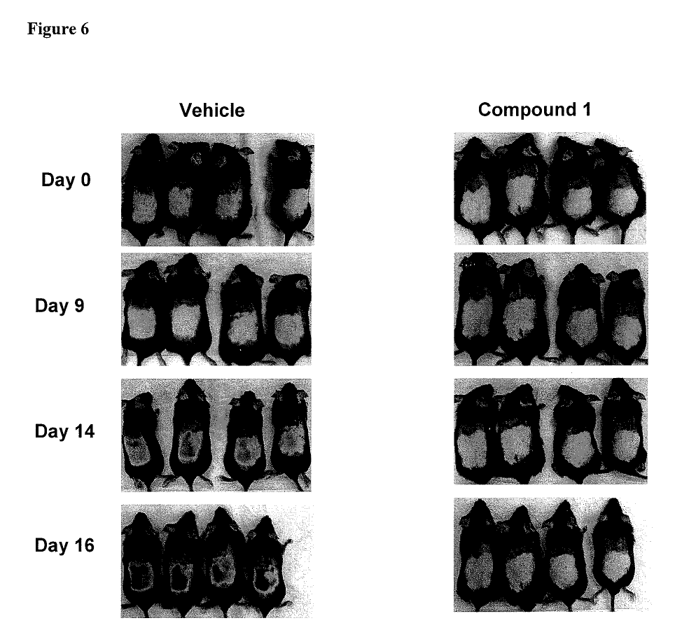

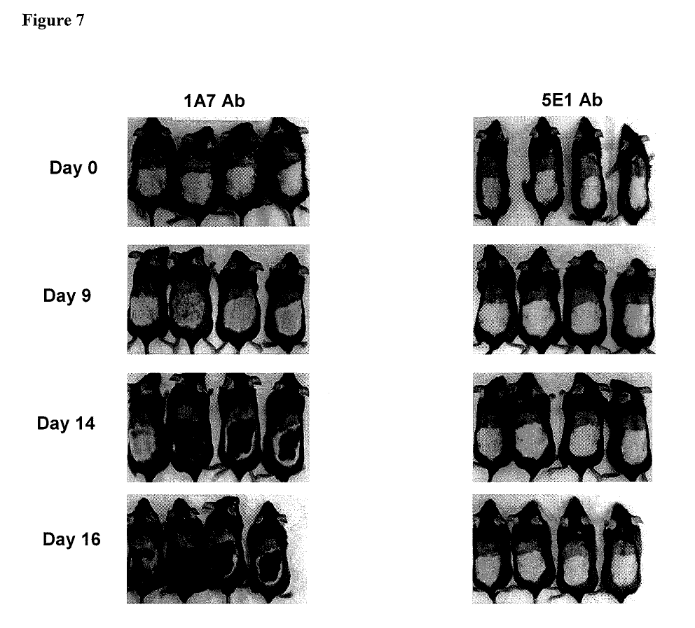

It was found that melanogeneis and hair re-growth post depilation is prevented by daily administration of 40 mg/kg Compound 1; hair re-growth post depilation is also prevented by BID administration of 6 mg/kg 5E1. See FIGS. 6 and 7.

Mice were depilated at 7 weeks of age, and on the day of depilation (day 0) daily dosing of either vehicle (30% HPBCD) or 40 mg/kg Compound 1 was begun. As additional controls, two groups dosed with either the anti-hedgehog monoclonal antibody (5E1) or its isotype control antibody (1A7) were included. Six mice were included in each treatment group. The hybridoma cell lines producing anti-hedgehog monoclonal antibody 5E1, and the isotype control antibody 1A7, were purchased from the Developmental Studies Hybridoma Bank at the University of Iowa. Both antibodies were dosed every other day at a concentration of 6 mg/kg via i.p. injection. The study was taken down on day 16 post depilation as hair had grown back on all of the vehicle and 1A7 treated mice, but not on the Compound 1 or 5E1 treated mice. To record hair growth throughout the study, pictures were taken of the same four mice from each group on days 0, 9, 14 and 16 post depilation (FIGS. 6 & 7).

### Example 7

### Inhibition of Gli-1 Induction

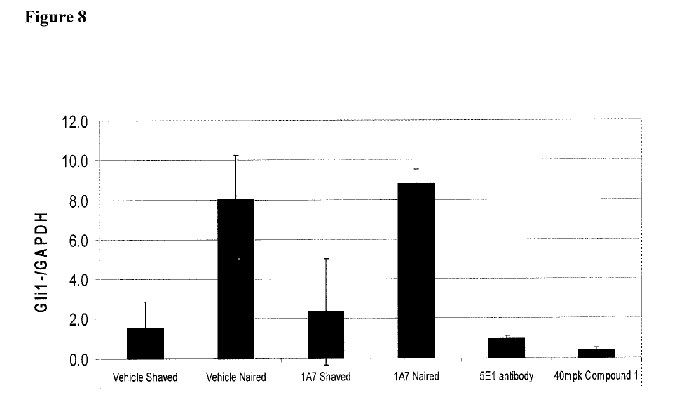

RT-PCR analysis from skin collected from the mice used in the hair re-growth study showed that both Compound 1 and 5E1 were able to inhibit Gli-1 induction as well as hair re-growth post depilation (FIG. 8).

### Example 8

### Inhibition of Hair Re-Growth Post the Onset of Natural Anagen

The ability of Compound 1 to inhibit hair re-growth post the onset of natural anagen was investigated. The results (not shown) demonstrated that, as in depilation-induced anagen, daily administration of Compound 1 but not vehicle prevented hair growth caused by the onset of natural anagen in C57BL/6 mice that were shaved while in telogen (7-12 weeks of age) and allowed to progress into anagen (12-16 weeks of age). Administration of Compound 1 was initiated when the first group of mice (in this case vehicle-treated mice) displayed signs of melanogenesis and continued for 3 weeks (until four of the six mice within that group (vehicle) grew back hair).

### INCORPORATION BY REFERENCE

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of ascertaining the inhibitory activity of a candidate inhibitor of the hedgehog pathway, comprising the steps of:
inducing an anagen phase in one or more hair follicles in a first mammal and a second mammal;
administering to said first mammal said candidate inhibitor;
measuring a detectable marker of hedgehog pathway activity in said first mammal, thereby obtaining a first detectable marker activity;
measuring said detectable marker of hedgehog pathway activity in said second mammal, thereby obtaining a second detectable marker activity; and
comparing said first detectable marker activity and said second detectable marker activity.

2. The method of claim 1, wherein said candidate inhibitor is administered systemically.

3. The method of claim 1, wherein the anagen phase is induced by chemical or physical depilation.

4. (canceled)

5. The method of claim 1, wherein the detectable marker is HhIP, Gli1, Gli2, Gli3, Ptc1, Ptc2, sonic hedgehog, indian hedgehog, or desert hedgehog.

6. The method of claim 1, wherein the detectable marker is measured by RT-PCR, in situ hybridization or immunohistochemistry.

7. The method of claim 1, wherein the mammal is a rodent or primate.

8. The method of claim 1, wherein the mammal is a mouse.

9. The method of claim 1, wherein the mammal is a C57BL/6 or C3HMCA mouse.

10. (canceled)

11. The method of claim 1, wherein the anagen phase is induced from about 7 weeks to about 12 weeks after said mammal is born.

12. A method of ascertaining the inhibitory activity of a candidate inhibitor of the hedgehog pathway, comprising the steps of:
artificially inducing an anagen phase in substantially all hair follicles in a section of skin of a first mouse and a second mouse;
administering to said first mouse said candidate inhibitor;
measuring a detectable marker of hedgehog pathway activity in said first mouse, thereby obtaining a first detectable marker activity;
measuring said detectable marker of hedgehog pathway activity in said second mouse, thereby obtaining a second detectable marker activity; and
comparing said first detectable marker activity and said second detectable marker activity.

13. (canceled)

14. The method of claim 12, wherein the anagen phase is artificially induced by chemical or physical depilation.

15. (canceled)

16. The method of claim 12, wherein the detectable marker is HhIP, Gli1, Gli2, Gli3, Ptc1, Ptc2, sonic hedgehog, indian hedgehog, or desert hedgehog.

17. The method of claim 12, wherein the detectable marker is measured by RT-PCR, in situ hybridization or immunohistochemistry.

18. The method of claim 12, wherein the mouse is a C57BL/6 or C3HMCA mouse.

19. (canceled)

20. The method of claim 12, wherein the anagen phase is artificially induced from about 7 weeks to about 12 weeks after said mouse is born.

21. A method of ascertaining the inhibitory activity of a candidate inhibitor of the hedgehog pathway, comprising the steps of:
providing a first non-human mammal and a second non-human mammal about a few days before the start of a naturally occurring anagen phase in one or more hair follicles or during a naturally occurring anagen phase of one or more hair follicles;
administering to said first non-human mammal said candidate inhibitor;
measuring a detectable marker of hedgehog pathway activity in said first non-human mammal, thereby obtaining a first detectable marker activity;
measuring said detectable marker of hedgehog pathway activity in said second non-human mammal, thereby obtaining a second detectable marker activity; and
comparing said first detectable marker activity and said second detectable marker activity.

22. (canceled)

23. The method of claim 21, wherein the detectable marker is HhIP, Gli1, Gli2, Gli3, Ptc1, Ptc2, sonic hedgehog, indian hedgehog, or desert hedgehog.

24. The method of claim 21, wherein the detectable marker is measured by RT-PCR, in situ hybridization or immunohistochemistry.

25. The method of claim 21, wherein said non-human-mammal is a mouse.

26. The method of claim 21, wherein the non-human mammal is a C57BL/6 or C3HMCA mouse.

27-31. (canceled)
